(19) United States
(12) Patent Application Publication
(10) Pub. No.: US 2013/0005833 A1
(43) Pub. Date: Jan. 3, 2013

(54) PHOTO-RESPONSIVE SUPRAMOLECULAR HYDROGELS

(76) Inventor: Yan Zhang, Nanjing (CN)

(21) Appl. No.: 13/505,839

(22) PCT Filed: Jul. 1, 2011
(86) PCT No.: PCT/CN2011/070084

§ 371 Date: May 3, 2012

(51) Int. Cl. A61K 47/42; C07K 5/093; C07K 5/083; C07K 5/09; C07K 5/087; C07K 5/072; C07K 5/062; C07K 5/068; C07K 7/06; C07K 5/103; C07K 4/00; C07K 5/065

(52) U.S. Cl. 514/773; 530/300; 530/331; 534/853; 530/330

(57) ABSTRACT

Compounds represented by general Formula I may form photo-responsive hydrogels that can be modulated between a non-viscous liquid state and a hydrogel. Formula I is:

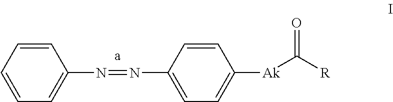

where a stereochemical conformation of double bond “a” is cis- or trans-, Ak is an alkylene group; and R represents a series of two or more naturally occurring or synthetic amino acid residues.

### FIELD

The present technology generally relates to compositions that reversibly gel (sol to gel or gel to sol) in response to different stimuli.

### SUMMARY

In one aspect, composition including a stimuli-responsive compound represented by Formula I, or a salt thereof is provided, where Formula I is:

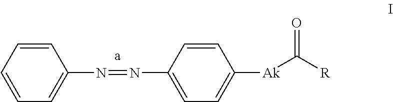

and where, a stereochemical conformation of double bond “a” is cis- or trans-; Ak is an alkylenyl group; and R represents a series of two or more naturally occurring or synthetic amino acid residues. In some embodiments, the compound of Formula I is represented more specifically by a Formula II, III, IV, or V; or a salt of Formula II, III, IV, or V:

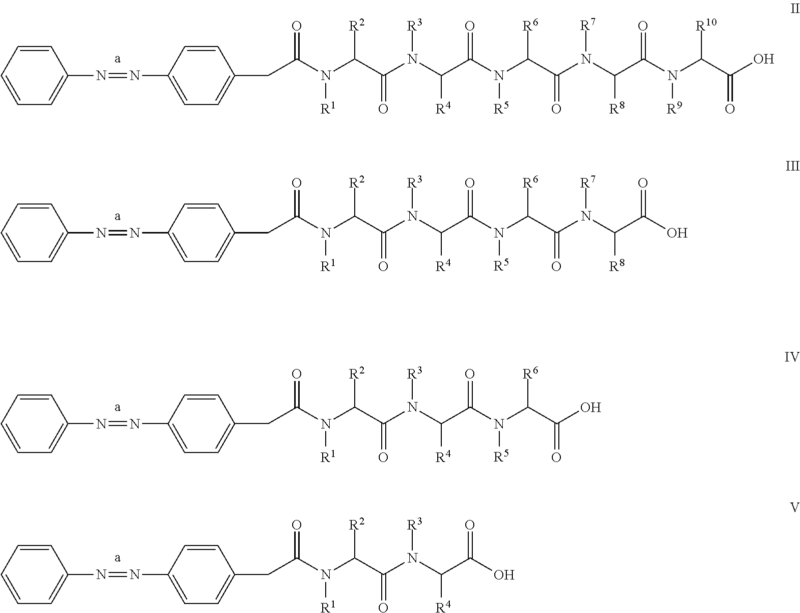

wherein a stereochemical conformation of double bond “a” is cis- or trans-, and R¹, R³, R⁵, R⁷, and R⁹ are independently absent or H. According to some embodiments, where any one of Rⁿ is H, Rⁿ⁺¹ is H, OH, SH, COOH, COOR, alkyl, alkenyl, alkynyl, aryl, heterocyclyl, heteroaryl, aralkyl, amine, or amide; and where any one of Rⁿ is absent, Rⁿ⁺¹ is alkylenyl and joins with the nitrogen of NR' to form a heterocycle. Rⁿ represents R¹, R³, R⁵, R⁷, and R⁹, where n is 1, 3, 5, 7, or 9.

In another aspect, a method includes exposing a non-viscous solution to visible light, wherein the non-viscous solution includes a composition including a compound of Formula I and water; and upon exposure of the non-viscous solution to visible light for a first sufficient period of time, the non-viscous solution forms a hydrogel. In some embodiments, the method also includes exposing the hydrogel to one or more of UV light, ultrasonication, or heat, wherein upon exposure to one or more of UV light, ultrasonication, or heat, for a second sufficient period of time, the hydrogel collapses to re-form the non-viscous solution.

In another aspect, a controlled release composition is provided including a pharmaceutically active agent and a composition including the compound represented by Formula I.

In another aspect, a process includes providing a non-viscous solution including a pharmaceutically active agent, a composition including the compound represented by Formula I, and water; and exposing the non-viscous solution to visible light for sufficient period of time for the composition to form a hydrogel; wherein the hydrogel is a controlled release pharmaceutical composition.

### BRIEF DESCRIPTION OF THE DRAWINGS

FIGS. 1A and B are graphs illustrating lowest concentration of hydrogelation for Azo-dipeptides, pH dependence (A), and salt dependence (B), according to the examples.

FIG. 2A is a compilation of circular dichroism (CD) spectra of hydrogels formed by Azo-X-Phe-Ala, according to the examples.

FIG. 2B is a compilation of circular dichroism spectra of hydrogels of Azo-D-Lys-D-Phe-D-Ala; Azo-D-Lys-Phe-Ala; Azo-Lys-D-Phe-D-Ala and Azo-Lys-Phe-Ala, according to the examples.

FIG. 3 is an ultraviolet (UV) spectrum of an Azo-Gln-Phe-Ala hydrogel before (solid line) and after (dashed line) UV irradiation, according to the examples.

FIG. 4 is a graph of the strain sweep test (A) and the frequency sweep test (B) of the gel formed by Azo-Lys-D-Phe-D-Ala before and after UV irradiation, according to the examples.

FIG. 5 is a graphical comparison of the release ratio of vitamin B12 with (squares) and without (dots) UV irradiation, according to the examples.

### DETAILED DESCRIPTION

In the following detailed description, reference is made to the accompanying drawings, which form a part thereof. The illustrative embodiments described in the detailed description, drawings and claims are not meant to be limiting. Other embodiments may be utilized, and other changes may be made, without departing from the spirit or scope of the subject matter presented here. The present technology is also illustrated by the examples herein, which should not be construed as limiting in any way.

In one aspect, stimuli-responsive hydrogels based on azobenzene-substituted short peptides are provided. As used herein, a short peptide is one having 20 or fewer amino acid residues. The short peptides appropriately linked to the conformational switch azobenzene, exhibit a switchable photo-response where hydrogels form in visible light and the hydrogels collapse under UV irradiation. The compounds may also exhibit pH and/or salt dependence.

According to some embodiments, the wavelength of the visible light is greater than about 400 nm. In some embodiments the wavelength of the visible light is from about 400 nm to about 790 nm. The intensity of the visible light may range from about 15 W to about 200 W. In some embodiments, the intensity of the visible light is about 100 W. According to some embodiments, the wavelength of the UV light is from about 10 to about 400 nm. In some embodiments the wavelength of the UV light is about 365 nm. The intensity of the UV light may range from about 300 W to about 600 W. In some embodiments, the intensity of the visible light is about 500 W.

In general, the stimuli-responsive hydrogels may be prepared from compounds that may be represented by Formula I, or a salt of the compound represented by Formula I:

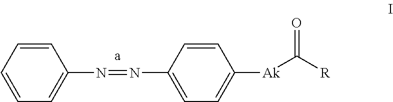

In Formula I, the double bond designated as “a” may be in either the cis- or trans-stereochemical conformation. The cis-/trans-isomers can alternatively be referred to as Z-/E-isomers, respectively. In Formula I, Ak is an alkylene group. In Formula I, R represents a series of two or more naturally occurring or synthetic amino acid residues.

In some embodiments of Formula I, the stereochemical conformation of double bond “a” is cis-. In other embodiments of Formula I, the stereochemical conformation of double bond “a” is trans-.

In some embodiments of Formula I, Ak is a C₁-C₁₀ alkylene group. In some embodiments, Ak is methylene, ethylene, or propylene.

In some embodiments of Formula I, R represents from 2 to 20 naturally occurring or synthetic amino acid residues. R can contain only naturally occurring amino acid residues, only synthetic amino acid residues, or a mixture of some naturally occurring amino acid residues and some synthetic amino acid residues. Examples of R groups can be Xaa₁₋₂, Xaa₁₋₃, Xaa₁₋₄, Xaa₁₋₅, Xaa₁₋₆, Xaa₁₋₇, Xaa₁₋₈, Xaa₁₋₉, Xaa₁₋₁₀, Xaa₁₋₁₁, Xaa₁₋₁₂, Xaa₁₋₁₃, Xaa₁₋₁₄, Xaa₁₋₁₅, Xaa₁₋₁₆, Xaa₁₋₁₇, Xaa₁₋₁₈, Xaa₁₋₁₉, or Xaa₁₋₂₀. Naturally occurring amino acids include the following (indicated by their full name and three- and one-letter abbreviations): Alanine (Ala, A); Arginine (Arg, R); Asparagine (Asn, N); Aspartic acid (Asp, D); Cysteine (Cys, C); Glutamic acid (Glu, E); Glutamine (Gln, Q); Glycine (Gly, G); Histidine (His, H); Isoleucine (Ile, I); Leucine (Leu, L); Lysine (Lys, K); Methionine (Met, M); Phenylalanine (Phe, F); Proline (Pro, P); Serine (Ser, S); Threonine (Thr, T); Tryptophan (Trp, W); Tyrosine (Tyr, Y); and Valine (Val, V). The amino acid residues can have various steriosomer configurations. For example, the amino acids can have the naturally occurring L- or unnatural D- configurations. Examples of synthetic amino acid residues include those amino acids that are chemically modified by substitutions which are not naturally occurring. The amino acid residues can include one or more non-standard amino acid residues such as lanthionine, 2-aminoisobutyric acid, dehydroalanine, gamma-aminobutyric acid, selenocysteine, hypusine, beta-alanine, ornithine, and citrulline. In some embodiments, R is represented by: Xaa₁Xaa₂Xaa₃Xaa₄Xaa₅Xaa₆Xaa₇Xaa₈Xaa₉Xaa₁₀. In such embodiments, Xaa₁ is a naturally occurring or synthetic amino acid. In other embodiments, each of Xaa₂, Xaa₃, Xaa₄, Xaa₅, Xaa₆, Xaa₇, Xaa₈, Xaa₉, and Xaa₁₀ is independently des-Xaa, or a naturally occurring or synthetic amino acid. In some embodiments, at least Xaa₆, Xaa₇, Xaa₈, Xaa₉, and Xaa₁₀ are des-Xaa. In other embodiments, each of Xaa₅, Xaa₆, Xaa₇, Xaa₈, Xaa₉, and Xaa₁₀ is des-Xaa. In other embodiments, each of Xaa₈, Xaa₉, and Xaa₁₀ is des-Xaa. As used herein, the prefix “des-”, as it is applied to a generic amino acid designator (Xaa) or a specific amino acid, indicates the absence of that particular group. For example, where each of Xaa₈, Xaa₉, and Xaa₁₀ is des-Xaa, each of Xaa₈, Xaa₉, and Xaa₁₀ is absent.

The stimuli-responsive compound may be more specifically represented by sub formulas of Formula I. For example, in some embodiments, the stimuli-responsive compound is represented by Formula II (having 5 amino acid residues), III (having 4 amino acid residues), IV (having 3 amino acid residues), or V (having 2 amino acid residues); or a salt of Formula II, III, IV, or V:

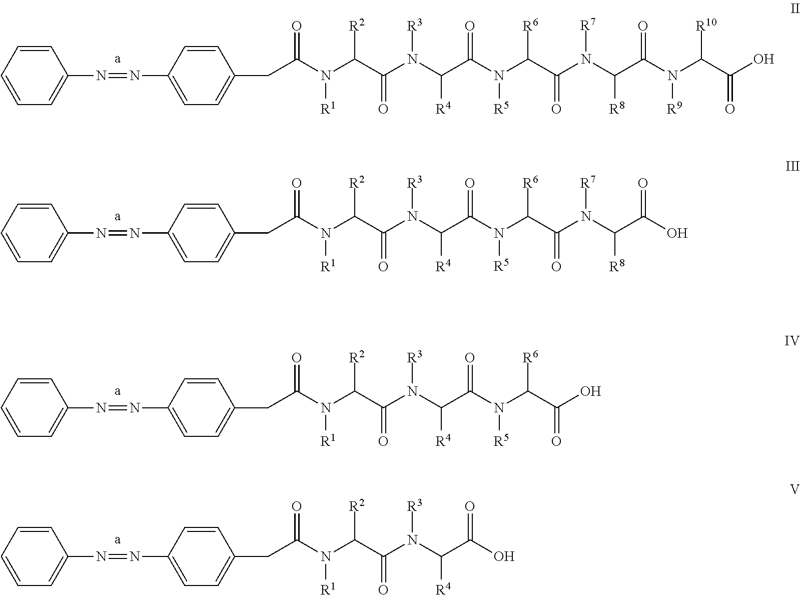

For Formulas II, III, IV, or V, a stereochemical conformation of double bond “a” is cis- or trans-; and each of R¹, R³, R⁵, R⁷, and R⁹ are independently absent or H. Where R¹, R³, R⁵, R⁷, or R⁹ (i.e. Rⁿ where n is 1, 3, 5, 7, or 9) is H, then the corresponding Rⁿ⁺¹ (i.e. R², R⁴, R⁶, R⁸ or R¹⁰) is H, OH, SH, COOH, COOR, alkyl, alkenyl, alkynyl, aryl, heterocyclyl, heteroaryl, aralkyl, amine, or amide. However, where any one or more of R¹, R³, R⁵, R⁷, or R⁹ is absent, R¹⁰ is an alkylenyl moiety that joins with the nitrogen of NRⁿ to form a heterocycle.

In some embodiments of the compounds of Formula II, III, IV, or V; R¹, R³, R⁵, R⁷ and R⁹ are H. In some such embodiments, R², R⁴, R⁶, R⁸ and R¹⁰ are independently H, C₁-C₈ alkyl, substituted C₁-C₈ alkyl, C₁-C₆ cycloalkyl, substituted C₁-C₆ cycloalkyl, phenyl, substituted phenyl, C₃-C₁₀ heterocyclyl, C₃-C₁₀ substituted heterocyclyl, C₃-C₁₀ heteroaryl, C₃-C₁₀ substituted heterocyclyl, or aralkyl.

In other embodiments, where R′, R³, R⁵, R⁷ and R⁹ are H, then R², R⁴, R⁶, R⁸ and R¹⁰ may independently be H, methyl, ethyl, n-propyl, iso-propyl, 2-methylprop-1-yl, 1-methylprop-1-yl, n-butyl, CH₂OH, CH(CH₃)(OH), CH₂SH, CH₂CH₂SH, CH₂CH₂SCH₃, CH₂(phenyl), CH₂(o-phenol), CH₂(m-phenol), CH₂(p-phenol), CH₂(indol-3-yl), CH₂C(O)OH, CH₂CH₂C(O)OH, CH₂C(O)NH₂, CH₂CH₂C(O)NH, CH₂(imidazole-5-yl), CH₂CH₂CH₂CH₂NH₂, CH₂CH₂CH₂NHC(NH)(NH₂), CH₂CH₂CH(OH)CH₂NH₂, CH₂CH₂CH₂CH₂NHCH₃, CH₂CH(COOH)COOH, CH₂OPO₃H₂, CH(CH₃)OPO₃H₂, CH₂CH₂OH, CH₂CH₂CH₂NH₂, CH₂CH₂CH₂NHC(O)NH₂, phenyl, CH(CH₃)COOH.

As noted above, one or more of R′, R³, R⁵, R⁷ and R⁹ may be absent. Accordingly, the nitrogen to which the absent R¹, R³, R⁵, R⁷, or R⁹ would otherwise have been bound, will join with the corresponding member of R², R⁴, R⁶, R⁸ or R¹⁰ to form a C₃-C₆ heterocycle. In other words where Rⁿ, where n is 1, 3, 5, 7, or 9 is absent, then Rⁿ⁺¹ joins with the nitrogen of NRⁿ to form a heterocycle. In some embodiments, such a heterocycle is prolinyl moiety.

The stimuli-responsive compositions may be responsive to changes in the wavelength of irradiating light on the composition, salt changes, or pH changes, or other stimuli that result in a physical changes of the composition. For example, as noted above, when a non-viscous composition containing one or more of the compounds of Formula I (or the sub-Formulas II, III, IV, or V) is exposed to a visible light source for a sufficient period of time, the composition gels to form a hydrogel. As used herein a non-viscous liquid is one which readily flows and does not maintain a shape. In contrast, as used herein, a hydrogel is a viscous fluid that does not flow readily and which retains a shape, such as that of a container in which the hydrogel is located. In some embodiments, the non-viscous composition has a first viscosity, and the hydrogel has a second viscosity, where the second viscosity is higher than the first viscosity.

Prior to forming the gel, the composition is a non-viscous liquid. After gelation, the hydrogel may be irradiated with UV light to cause the hydrogel to collapse and re-form the non-viscous liquid. In some embodiments, the initial non-viscous composition has a first viscosity, the hydrogel has a second viscosity, and the collapsed liquid has a third viscosity, where a) the second viscosity is higher than the first viscosity, and b) the third viscosity is lower than the second viscosity. The first viscosity and the third viscosity can be the same or different. For example, the third viscosity can be higher than the first viscosity, or the third viscosity can be lower than the first viscosity. The pH of the composition and the salt content of the system may also impact gelation in a manner ranging from preventing gelation to increasing the speed at which the hydrogels form and collapse in response to an applied photostimulus.

Although not intending to be limited by a particular theory, the formation of the hydrogel is believed to be due to the cis-/trans-isomerization of the azobenzene moiety when exposed to irradiation of different wavelengths. Under irradiation by light of visible wavelengths, the trans-configuration is preferred and the gels form. Under irradiation by light of UV wavelengths, the cis-conformation is preferred and the hydrogel collapses. The cis-trans conformational switch of the peptides described herein was at the N-terminus of the short peptides. A representative structure of such compounds that are stimuli responsive, and which exhibit a conformational switch is shown Scheme 1, where the left-side structure has the N═N double bond in a “trans” conformation, and the right-side structure has the N═N double bond in a “cis” conformation. The alkylene spacer between the phenyl group and the carbonyl group (indicated by the arrow in Scheme 1; depicted as a methylene group for illustration) provides for significant improvement in the ability of the substituted short peptides to dissolve in water without organic solvents to form the hydrogels.

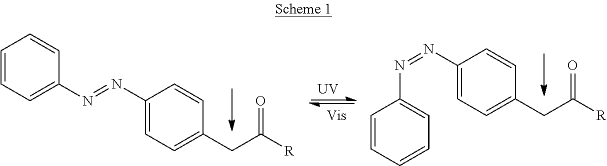

Without being bound by theory, it is believed that the conformation-switchable azobenzene is a contributor to the hydrophobic interaction in the self-assembly system through effective intermolecular π-π stacking of the phenyl rings on the azobenzene moiety when the azobenzene is in the trans-configuration. Meanwhile, the peptide backbone of the structure provides for hydrogen bonding sites of intermolecular hydrophilic sites. When the balance between the π-π stacking interaction and hydrogen bonding is achieved, the self-assembled system forms three dimensional fibrous networks as a gel with water (i.e. a hydrogel). See Scheme 2.

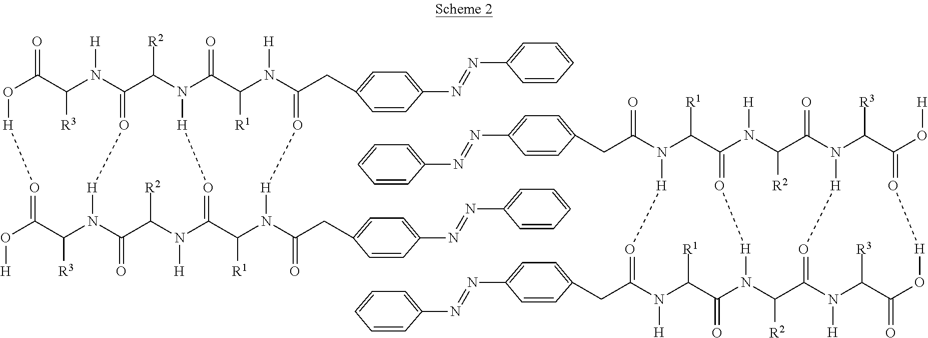

However, under appropriate conditions, conformational switching of the azobenzene moiety's N═N double bond from trans- to cis-changes the relative position of the phenyl rings, causing for an increase in intermolecular distances, which weakens the π-π stacking effects (Scheme 3). As the ability to maintain the π-π stacking is diminished as more of the cis-conformer is formed, the fibrous hydrogel network begins to fail and a phase change from hydrogel to non-viscous solution is observed. See Scheme 3.

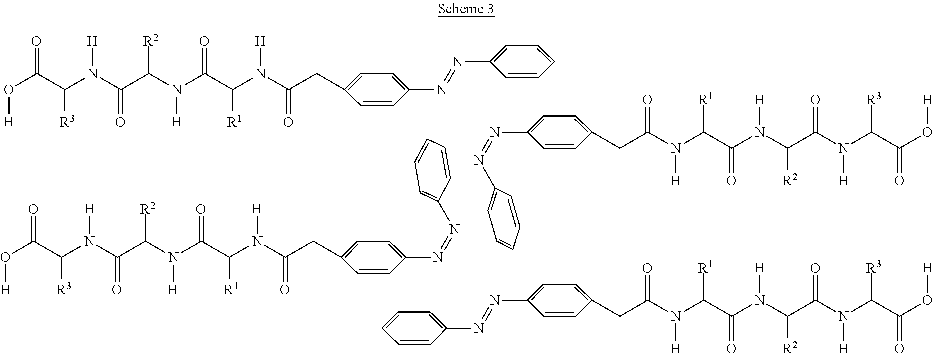

It is noteworthy that in the examples provided above and below, the conformation switch of the peptides is located at the N-terminus of the short peptides. Instead of using (E)-4-(phenyldiazenyl)benzoic acid to couple with the N-terminal amino group of the amino acids, (E)-2-(4-(phenyldiazenyl)phenyl)acetic acid was used as the N-terminal substitution group of the short peptides. This subtle structural difference results in the improved ability of the substituted short peptides to dissolve in water without the use of additional organic solvents.

Thus, in another aspect, a method is provided including exposing a non-viscous solution to visible light, wherein the non-viscous solution includes a compound of Formula I, II, III, IV, and/or V and water. Such exposure is maintained for a sufficient period of time to allow the non-viscous solution to form a hydrogel. According to some embodiments, the sufficient period of time is from about 1 hour to 5 days. For example, the sufficient period of time may be from about 1 hour to 5 days, from about 10 hours to about 5 days, or from about 1 day to about 3 days. In some embodiments, the non-viscous composition has a first viscosity, and the hydrogel has a second viscosity, where the second viscosity is higher than the first viscosity.

To form a hydrogel a minimum amount of compounds in the trans-configuration should be present. If too few are in the trans-configuration π-π stacking is inefficient and the hydrogel will not have sufficient structural integrity. Accordingly, in some embodiments of the method, upon the exposing the composition to visible light, greater than about 90% of the compounds of Formula I (or sub-Formulas II, III, IV, or V), or the salt thereof in the composition are in the trans-configuration. In other embodiments of the method, after exposing of the composition to visible light, greater than about 95% of the compounds of Formula I (or sub-Formulas II, III, IV, or V), or the salt thereof in the composition are in the trans-configuration.

The method may also include exposing a hydrogel to one or more of UV light, ultrasonication, or heat for a sufficient period of time to cause the hydrogel to collapse and form a collapsed non-viscous solution. Exposure of the hydrogels to UV light will cause a conformation switch of at least some of the compounds in the trans-configuration to the cis-configuration. According to some embodiments, the sufficient period of time is from about 10 seconds to about 1 day. For example, the sufficient period of time may be from about 3 minutes to about 1 day, about 3 minutes to about 10 hours, from about 3 minutes to about 1 hour, from about 10 minutes to 30 minutes. In some embodiments of the method, upon exposing the hydrogel to UV light, the compounds of Formula I (or sub-Formulas II, III, IV, or V), or the salt thereof in the composition are randomly distributed among the cis- and trans-configurations. In other embodiments of the method, after exposing the composition to UV light, greater than about 10% of the compounds of Formula I, or the salt thereof in the composition are in the cis-configuration. In other embodiments of the method, after exposing the composition to UV light, greater than about 20%, 30%, 40%, 50%, 60%, 80%, or 90% of the compounds of Formula I, or the salt thereof in the composition are in the cis-configuration. In other embodiments of the method, after exposing the composition to UV light, greater than about 87% of the compounds of Formula I, or the salt thereof in the composition are in the cis- configuration. In some embodiments, the initial non-viscous composition has a first viscosity, the hydrogel has a second viscosity, and the collapsed liquid has a third viscosity, where a) the second viscosity is higher than the first viscosity, and b) the third viscosity is lower than the second viscosity. The first viscosity and the third viscosity can be the same or different. For example, the third viscosity can be higher than the first viscosity, or the third viscosity can be lower than the first viscosity.

In another aspect, the compositions may be controlled release compositions for the delivery of pharmaceutically active agents. Such controlled release compositions may be prepared as a non-viscous solution of a compound of Formula I, II, III, IV, and/or V with water and the pharmaceutically acceptable active agent. The non-viscous solution may then be gelled by exposure of the solution to visible light to form a hydrogel. The hydrogel may then be used as a controlled release composition. Without being bound by theory it is believed that such hydrogels form a matrix that causes the pharmaceutically active agent to have to diffuse through the gel before release of the agent from the composition may be achieved. Thus, the matrix prevents immediate release (e.g. delays and/or prolongs release) of the agent.

Such compositions may be administered to a subject in the hydrogel form or in the liquid form that is then gelled in vivo. For example, when the solution is administered, it may be via surgical placement in a subject or via parenteral routes, followed by gellation with visible light. When the hydrogel is administered, it may be via oral or parenteral routes. The controlled release of the pharmaceutically active agent may then be controlled by diffusion, irradiation with UV light, changes in pH, or changes in salt concentration. Where the administration is by diffusion, the pharmaceutically active agent is allowed to diffuse from the hydrogel by the influence of a concentration gradient without external effects on the hydrogel. Where the administration includes the use of the application of UV irradiation, the hydrogel is irradiated by UV light to cause collapse and subsequent release of the pharmaceutically active agent. Where the administration includes changes in pH or salt concentration, the hydrogel may be formed at a pH or salt concentration different from that found physiologically in the subject to which the composition is to be administered. When administered, the change in pH or salt concentration may then cause collapse of the hydrogel and subsequent release of the pharmaceutically active agent.

According to various embodiments, the pharmaceutically active agent includes, but is not limited to, e.g., antimuscarinics, prostaglandin analogues, proton pump inhibitors, aminosalycilates, corticosteroids, chelating agents, cardiac glycosides, phosphodiesterase inhibitors, thiazides, diuretics, carbonic anhydrase inhibitors, antihypertensives, anti-cancer agents, anti-depressants, calcium channel blockers, analgesics, opioid antagonists, antiplatelets, anticoagulants, fibrinolytics, statins, adrenoceptor agonists, beta blockers, antihistamines, respiratory stimulants, micolytics, expectorants, benzodiazepines, barbiturates, anxiolytics, antipsychotics, tricyclic antidepressants, 5HT₁ antagonists, opiates, 5HT₁ agonists, antiemetics, antiepileptics, dopaminergics, antibiotics, antifungals, anthelmintics, antivirals, antiprotozoals, antidiabetics, insulin, thyrotoxin, female sex hormones, male sex hormones, antioestrogen, hypothalamics, pituitary hormones, posterior pituitary hormone antagonists, antidiuretic hormone antagonists, bisphosphonates, dopamine receptor stimulants, androgens, non-steroidal anti-inflammatorys, immuno suppressant local anaesthetics, sedatives, antipsioriatics, silver salts, topical antibacterials, vaccines, and vitamins. In some embodiments, the composition may include, but is not limited to, e.g., vitamin B12, Ibuprofen, Aspirin, Penicillin G, Cephradine, Taxol, Carmustine, Lanreotide, Amoxicillin, Ganirelix, Pamidronate, or Chlorambucil.

A method of preparing a controlled release composition thus includes, providing a non-viscous solution comprising a pharmaceutically active agent, a compound of Formula I, II, III, IV, or V, and water; and exposing the non-viscous solution to visible light for sufficient period of time for the composition to form a hydrogel. Such a hydrogel may be used as a controlled release pharmaceutical composition as described above. In some embodiments, where the active agent is vitamin B12, a concentration of greater than about 10 mg/ml may be used. For example, the concentration may range from about 10 mg/ml to about 50 mg/ml.

As used herein, the following useage of terms shall apply unless otherwise indicated.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

As used herein, “thiols” are compounds with an “SH” functional group represented by R—SH where R may be H, an alkyl, or aryl group.

Alkyl groups include straight chain, branched chain, or cyclic alkyl groups having from 1 to 20 carbon atoms or, in some embodiments, from 1 to 12, 1 to 8, 1 to 6, or 1 to 4 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. Where the term haloalkyl is used, the alkyl group is substituted with one or more halogen atoms.

Alkylenyl groups include di-radical straight chain, or branched chain alkyl groups having from 1 to 20 carbon atoms or, in some embodiments, from 1 to 12, 1 to 8, 1 to 6, or 1 to 4 carbon atoms. Alkylenyl groups are those which are alkyl based but which bridge two other moieties with two attachment. Examples of straight chain (“n”) alkylenyl groups include those with from 1 to 8 carbon atoms such as methyleneyl (—CH₂—), ethyleneyl (—CH₂CH₂—), n-propyleneyl (—CH₂CH₂CH₂—), n-butyleneyl (—CH₂CH₂CH₂CH₂—), n-pentyleneyl (—CH₂CH₂CH₂CH₂CH₂—), n-hexyleneyl (—CH₂CH₂CH₂CH₂CH₂CH₂—), n-heptyleneyl (—CH₂CH₂CH₂CH₂CH₂CH₂CH₂—), and n-octylenyl groups (—CH₂CH₂CH₂CH₂CH₂CH₂CH₂CH₂—). In some embodiments, the alkylenyl group is —CH₂CH₂CH₂—, or a propylenyl group.

Alkenyl groups include straight and branched chain alkyl and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 10 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Alkenyl groups may be substituted or unsubstituted.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 10 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others. Alkynyl groups may be substituted or unsubstituted.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3, or 4 heteroatoms. In some embodiments, heterocyclyl groups include 3 to 20 ring members, whereas other such groups have 3 to 6, 10, 12, or 15 ring members. Heterocyclyl groups encompass unsaturated, partially saturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]-dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups.” Heterocyclyl groups may be substituted or unsubstituted. Heterocyclyl groups include, but are not limited to, pyrrolidinyl, pyrrolinyl, imidazolyl, imidazolinyl, imidazolidinyl, piperidinyl, piperazinyl, morpholinyl, pyrrolyl, pyrazolyl, pyrazolidinyl, tetrahydropyranyl, thiomorpholinyl, pyranyl, triazolyl, tetrazolyl, furanyl, tetrahydrofuranyl, oxazolyl, isoxazolyl, thiazolyl, pyridinyl, pyrimidinyl, pyridazinyl, pyrazinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, quinazolinyl, benzotriazolyl, 2,3-dihydrobenzo[1,4]dioxinyl, and benzo[1,3]dioxolyl groups. Representative substituted heterocyclyl groups may be mono-substituted or substituted more than once, such as, but not limited to, pyridinyl or morpholinyl groups, which are 2-, 3-, 4-, 5-, or 6-substituted, or disubstituted with various groups as defined above, including, but not limited to, alkyl, oxo, carbonyl, amino, alkoxy, cyano, and/or halo.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Aryl, or arene, groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 20 carbon atoms, 7 to 14 carbon atoms or 7 to 10 carbon atoms. Substituted aralkyl groups can be substituted at the alkyl, the aryl or both the alkyl and aryl portions of the group. Representative aralkyl groups include but are not limited to benzyl and phenethyl groups and fused (cycloalkylaryl)alkyl groups such as 4-ethyl-indanyl. Representative substituted aralkyl groups can be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, heterocyclyl groups include 3 to 20 ring members, whereas other such groups have 3 to 6, 3 to 10, 3 to 12, or 3 to 15 ring members. Heterocyclyl groups encompass unsaturated, partially saturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups can be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups can be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups can be substituted at the alkyl, the heterocyclyl or both the alkyl and heterocyclyl portions of the group. Representative heterocyclyl alkyl groups include, but are not limited to, 4-ethyl-morpholinyl, 4-propylmorpholinyl, furan-2-yl methyl, furan-3-yl methyl, pyridine-3-yl methyl, tetrahydrofuran-2-yl ethyl, and indol-2-yl propyl. Representative substituted heterocyclylalkyl groups can be substituted one or more times with substituents such as those listed above.

As used herein, a pyrrolidinyl moiety is based upon the pyrolindinyl group of the amino acid proline. The moiety may be unsubstituted or substituted and has the general formula:

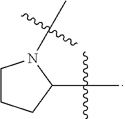

In general, “substituted” refers to a group, as defined above (e.g., an alkyl or aryl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, carbonyls(oxo), carboxyls, esters, urethanes, thiols, sulfides, sulfoxides, sulfones, sulfonyls, sulfonamides, amines, isocyanates, isothiocyanates, cyanates, thiocyanates, nitro groups, nitriles (i.e., CN), and the like.

The present technology, thus generally described, will be understood more readily by reference to the following examples, which are provided by way of illustration and are not intended to limit the present technology.

### EXAMPLES

### Example 1

Synthesis of the azobenzene substituted peptides. The azobenzene substituted short peptides were synthesized by solid phase peptide synthesis from 2-chlorotrityl chloride resin and the corresponding Fmoc-protected amino acids. For example, nitrosobenzene (1 eq) was coupled with ethyl 2-(4-aminophenyl)acetate (1 eq.) in acetic acid at room temperature to obtain ethyl 2-(4-(phenyldiazenyl)phenyl)acetate. The ethyl 2-(4-phenyldiazenyl)phenyl)acetate was then hydrolyzed in 1N KOH/MeOH to yield (4-phenylazophenyl)acetic acid.

The azobenzene substituted peptides were then prepared according to the following general procedure using solid phase peptide synthesis: 2-chlorotrityl chloride resin (1 g, 0.8-1.5 mmol/g) was suspended in 10 mL dichloromethane (DCM) for 5 min and filtered. To this was added a solution of Fmoc-amino acid (1.5 mmol; Fmoc is fluorenylmethyloxycarbonyl) and diisopropylethylamine (DIEA, 3 mmol) dissolved in 10 mL DCM. The mixture was then shaken for 30 minutes, filtered, and the resin washed with DMF (2×2 min). A mixture of DCM/MeOH/DIEA (80:15:5; 10 ml) was then added to the resin and repeated. The resin was then washed with dimethylformamide (DMF) (3×10 mL). A mixture of Fmoc-amino acid/HOBT (O-(1H-benzotriazol-1-yl)-N,N,N′, N′-tetramethyluronium hexafluorophosphate)/TBTU (2-(1H-benzotriazole-1-yl)-1,1,3,3-tetramethyluronium tetrafluoroborate)/DIEA (4.5 mmol/4.5 mmol/4.5 mmol/ 9 mmol) dissolved in 8 mL DMF was added to the resin, and shaken for reaction until a Kaiser Test showed the absence of free amino groups. Removal of the Fmoc was accomplished by washing with 20% piperidine in DMF (3×2 min), and further washing the resin with DMF (5×2 min; coupling steps). The coupling steps were then repeated to obtain the desired amino acid coupled to the resin.

After (4-Phenylazophenyl)acetic acid was coupled to the N-terminus of the peptides on the resin, the resultant azobenzene-substituted peptides were cleaved from the resin using trifluoroacetic acid (TFA). After removal of the TFA under vacuum, the crude oily product was further dispersed in ethyl ether followed by centrifugation to yield an amorphous powder. Purity of the substituted peptide products was greater than 95%, according to HPLC analysis. The azopeptides were then also characterized by ¹H NMR or electrospray mass spectrometry (ESI). The prepared azopeptides have the structure of Formula I, where Ak is a methylene (CH₂) group.

### Example 2

Preparation of Hydrogels. A homogeneous solution of azobenzene-substituted short peptides was prepared in a glass vial via pH adjustment and heating. Upon cooling to room temperature, a hydrogel formed for Azo-Gln-Phe-Ala; Azo-Lys-Phe-Ala; Azo-Leu- Phe-Ala; Azo-D-Lys-D-Phe-D-Ala; and Azo-Arg-Phe-Ala. However, Azo-Lys-D-Phe-D-Ala and Azo-D-Lys-Phe-Ala, sonification was necessary to facilitate the hydrogelation. Hydrogels of Azo-Ser-Phe-Ala; Azo-Gln-Tyr-Ala; Azo-Glu-Tyr-Ala; and Azo-Glu-Phe-Ala were prepared by acidifying the basic solution to the pH shown in Table 1.

TABLE 1
Hydrogelation conditions for azobenzene substituted tripeptides
and time needed for initial photo-responses

| | | | Conc. | | |
|---|---|---|---|---|---|
| No. | Azo-tripeptide | pH | (mg/mL)^{a} | T (° C.)^{b} | tᵢₙᵢₜ(min)^{c} |
| 1 | Azo-Gln-Phe-Ala | 7.8 | 3.7 | 65 | 3 |
| 2 | Azo-Glu-Phe-Ala | 4.7 | 4.2 | 85 | —^{d} |
| 3 | Azo-Leu-Phe-Ala | 8.6 | 4.7 | 71 | 15 |
| 4 | Azo-Gly-Phe-Ala | 7.5 | 7.2 | 48 | 5 |
| 5 | Azo-Arg-Phe-Ala | 1.6 | 2.8 | 47 | 8 |
| 6 | Azo-Ser-Phe-Ala | 3.7 | 3.2 | 54 | —^{d} |
| 7 | Azo-Gln-Tyr-Ala | 3.1 | 2.5 | 81 | — |
| 8 | Azo-Glu-Tyr-Ala | 3.0 | 3.0 | 69 | — |
| 9 | Azo-Lys-Phe-Ala | 1.6 | 3.1 | 59 | 30 |
| 10 | Azo-Lys-D-Phe-D-Ala | 1.2 | 6.8 | 42 | 7 |
| 11 | Azo-D-Lys-D-Phe-D-Ala | 1.6 | 3.0 | 60 | 15 |
| 12 | Azo-D-Lys-Phe-Ala | 1.4 | 6.8 | 39 | 4 |

^{a}Lowest concentration to gel water at specific pH;
^{b}temperature for gel to solution phase transition;
^{c}time needed for initial collapse of the gel upon UV irradiation;
^{d}no obvious change upon UV irradiation

### Example 3

Test on pH and salt effects on gelation properties. pH effect: Azo-dipeptides were mixed with a small quality of water and the mixture were adjusted to the desired pH with 1M HCl or NaOH, then heated to about 80-90° C. to obtain a clear solution. A yellow hydrogel formed upon cooling to room temperature. The lowest concentration for hydrogelation at a specific pH was obtained by successive dilution of the hydrogel using water, at the same pH that of the hydrogelation, to the point when hydrogel could not form.

Salt effect: Buffers at different pHs (5 and 6) and different concentrations (0.05M, 0.1M, 0.2M and 0.5M) were prepared by mixing Na₂HPO₄ and NaH₂PO₄ with different concentration. The azo-dipeptides were mixed with a small quality of buffer and heated to about 80-90° C. to obtain a clear solution. Yellow hydrogels formed upon cooling to room temperature. The lowest concentration for hydrogelation at specific pH was obtained by successive dilution of the hydrogel by the buffer with the same concentration of salt to the point when the hydrogel no longer formed.

### Example 4

Photo-response tests on the hydrogels. The light source for the UV irradiation was a high pressure mercury lamp (500 w) with a filter to cut off visible light. A control experiment was performed in parallel using the same hydrogel in a tin foil covered glass vial to exclude the possibility of response induced by any external stimulus other than light irradiation. The photo-response of the hydrogels was confirmed by observation of the gel to solution phase change of the sample under irradiation, but not on the sample of negative control. In one example, ¹H NMR of Azo-Lys-D-Ala-D-Ala in d-MeOH was used to confirm that after UV irradiation for 2 minutes 11% of the compound was in the cis-configuration, and after 10 minutes, 51% was in the cis-configuration.

### Example 5

Rheology test on the hydrogel before and after UV irradiation. All rheological measurements were performed using an ARES-G2 Rheometer with a cone and a plate (25 mm diameter plate and 0.0999 rad cone angle), and a gap opening between the cone and the plate of 0.0282 mm. The dynamic strain sweep test was carried out at 6.282 rad/s, and then dynamic frequency sweep test was investigated at critical strain, which was determined from storage-strain profile.

### Example 6

Hydrogelation properties of azobenzene substituted dipeptides. Different types of amino acid residues have been used in the combination of dipeptides to obtain information about the relative hydrogelation ability of the motifs in the azobenzene-substituted short peptides. Representative amino acid residues in the dipeptides include Phe (phenylalanine) and Tyr (tyrosine) which have aromatic side chains, Arg (arginine) and Lys (lysine) which have cationic side chains, Glu (glutamic acid) which contains an anionic side chain, Ser (serine) and Gln (glutamine) which have hydrophilic side chains and Ala (alanine) which contains an aliphatic side chain. The combination of the amino acids resulted in the azo-dipeptides shown in Table 2.

TABLE 2
Hydrogelation conditions for azobenzene
substituted dipeptides

| | | | C ^{a} |
|---|---|---|---|
| No. | Azo-XX | pH | (mg/mL) |
| 13 | Azo-Phe-Glu | 2.1 | 8.5 |
| 14 | Azo-Glu-Phe | 2.5 | 5.0 |
| 15 | Azo-Phe-Ser | 2.0 | 4.3 |
| 16 | Azo-Ser-Phe | 5.7 | 2.0 |
| 17 | Azo-Phe-Ala | 7.8 | 3.2 |
| 18 | Azo-Ala-Phe | 9.5 | 3.5 |
| 19 | Azo-Phe-Phe | 9.7 | 2.8 |
| 20 | Azo-Phe-Tyr | 8.6 | 5.8 |
| 21 | Azo-Tyr-Ala | 3.0 | 5.0 |
| 22 | Azo-Ala-Tyr | 5.1 | 2.5 |
| 23 | Azo-Tyr-Tyr | 4.8 | 0.8 |
| 24 | Azo-Glu-Tyr | 4.8 | 1.9 |
| 25 | Azo-Gln-Tyr | 4.8 | 1.9 |
| 26 | Azo-Gln-Gln | —^{b} | — |
| 27 | Azo-Gln-Ala | 2.5 | 6.0 |
| 28 | Azo-Glu-Ala | 2.4 | 4.2 |
| 29 | Azo-Arg-Ala | — | — |
| 30 | Azo-Arg-Phe | — | — |
| 31 | Azo-Arg-Gln | — | — |
| 32 | Azo-Ser-Ala | — | — |
| 33 | Azo-Lys-Ala | — | — |
| 34 | Azo-Arg-Lys | — | — |
| 35 | Azo-Glu-Lys | — | — |
| 36 | Azo-Tyr-Lys | 2.2 | 7.8 |

^{a} lowest concentration of hydrogelation at the corresponding pH;
^{b}unable to form hydrogel under tested conditions

Typical conditions for different azodipeptides to gel water were also listed in Table 2. From Table 2 it will be observed that the hydrogelation properties of azobenzene-substituted dipeptides are motif-dependant. The amino acid side chain plays a role in the hydrogelation process. Azo-dipeptides with aromatic amino acid residues such as Phe and Tyr readily form hydrogels. The combination of Phe with various types of amino acids including anionic Glu, hydrophilic Ser, aliphatic Ala and aromatic Phe or Tyr could all result in hydrogelators. The additional hydrophobic interaction induced by the aromatic side chain is favorable for the self-assembling of the azo-dipeptides. In comparison, azo-dipeptides with cationic amino acid residues such as Arg and Lys seemed to be either soluble or totally insoluble in water and therefore showed poor hydrogelation ability. It is also noteworthy that the distance between the Phe or Tyr residue and the N-terminal azobenzene influences the pH and concentration required for the hydrogelation of corresponding azo-dipeptides. Comparison of the gelation conditions for Azo-Phe-Glu to Azo-Glu-Phe; Azo-Phe-Ser to Azo-Ser-Phe; and Azo-Tyr-Ala to Azo-Ala-Tyr clearly shows a consistent change including higher pH and lower concentration required for hydrogelation. This indicates that hydrophobic interactions induced by the aromatic side chain of Phe or Tyr are more effective when Phe or Tyr is located further from the hydrophobic head of the substituted dipeptides. Such an enhanced hydrophobic interaction could lead to higher pH required for hydrogelation. Adjustment of the hydrogelation conditions for azodipeptides may be effected by the insertion of aromatic amino acid residues at appropriate positions.

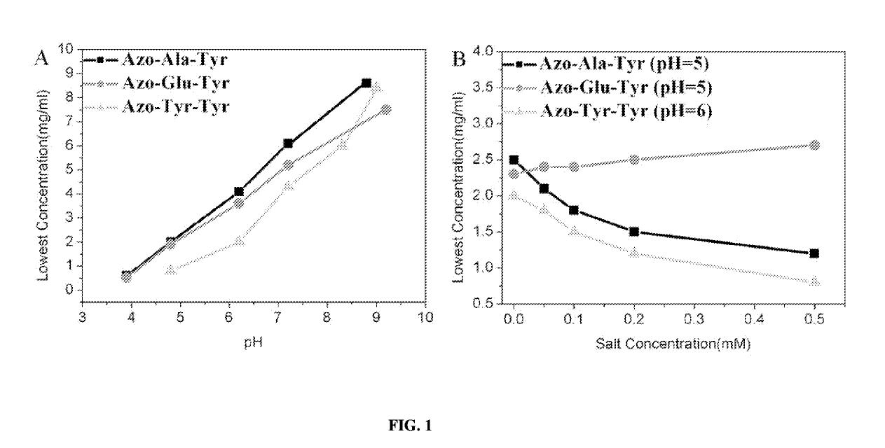

For the azo-dipeptides with Phe residues, hydrogelation occurred at a specific pH as listed in Table 2. At pH values lower than the listed pH for hydrogelation, the dipeptides precipitated out of solution, and could not be re-dissolved, even upon heating. However, azo-dipeptides with Tyr residues showed hydrogelation ability over a broad pH range. FIG. 1A illustrates the effect of pH on the lowest concentration required for hydrogelation for three dipeptides containing Tyr. The lowest concentration for hydrogelation is less than about 1% at pH of about 4. Solubility of the dipeptides increases with the increase in pH, therefore the concentration needed for gelation showed consistent increase with increasing pH.

The salt effect was also studied on these three azodipeptides hydrogelators. As shown in FIG. 1B, for Azo-Ala-Tyr and Azo-Tyr-Tyr, using a buffer of higher concentration of Na₂HPO₄/NaH₂PO₄ decreased the concentration required for hydrogelation. FIGS. 1A and 1B indicate that electrostatic interactions presented in the gel matrix could enhance the intermolecular interaction between the short peptides. However, for the dipeptide Azo-Glu-Tyr, which contains an anionic Glu, the salt effect was almost negligible compared with Azo-Ala-Tyr and Azo-Tyr-Tyr.

### Example 7

Hydrogelation properties of azobenzene substituted tripeptides. Azobenzene substituted tripeptides with Phe or Tyr between two other amino acid residues were prepared and their hydrogelation properties were studied. As shown in Table 1, insertion of Phe in between the two amino acids of the azodipeptides exhibits a regulatory role in hydrogelation as discussed above. Azo-Gln-Phe-Ala gel water at higher pH and lower concentration compared to Azo-Gln-Ala. Insertion of Phe in Azo-Arg-Ala; Azo-Ser-Ala; and Azo-Lys-Ala allowed the resulting tripeptides to gel water under acidic conditions (entry 5, 6 and 9), in contrast to the absence of gel formation as dipeptides. Insertion of Tyr instead of Phe provided for a similar effect on the regulation of the gelation properties. The tripeptides with cationic Arg or Lys residues (entries 5 and 9-12) gelled water under quite acidic conditions. It is noteworthy that the configuration of amino acid residues in the tripeptides showed slight effect on the hydrogelation properties of these tripeptides. The tripeptides with amino acids in uniform configurations such as Azo-Lys-Phe-Ala and Azo-D-Lys-D-Phe-D-Ala could form gel at lower concentrations than Azo-Lys-D-Phe-D-Ala and Azo-D-Lys-Phe-Ala which have both D- and L-amino acids in the tripeptide chains. Besides, lower phase transition temperature of gels formed by Azo-Lys-D-Phe-D-Ala or Azo-D-Lys-Phe-Ala than gels by Azo-Lys-Phe-Ala or Azo-D-Lys-D-Phe-DAla indicated weaker intermolecular strength.

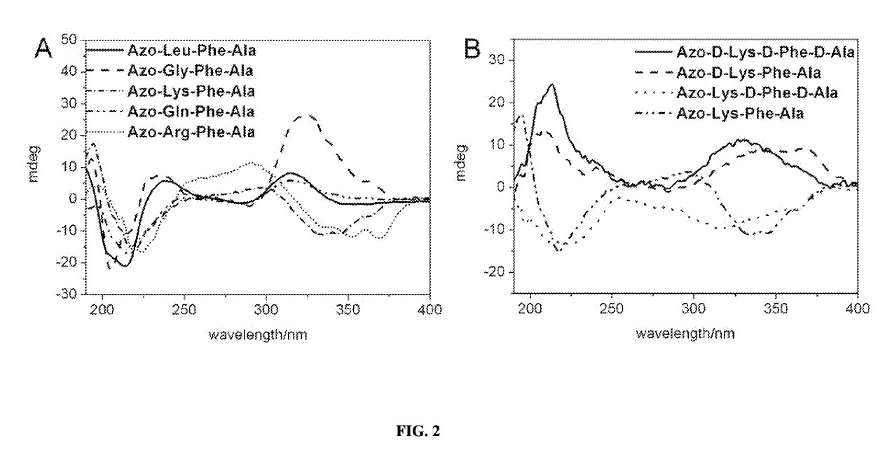

Circular dichroism (CD) spectra of the hydrogels is provided in FIG. 2. In FIG. 2A, the sharp peak at about 194 nm and the trough at about 214 nm indicate that the hydrogelators were mainly assembled in β sheet-like superstuctures in the gelled state. The peak at about 330 nm is due to π-π transitions of the cis-azobenzene group. CD spectra of the hydrogels formed by Azo-D-Lys-D-Phe-D-Ala and Azo-Lys-Phe-Ala showed symmetric signals as shown in FIG. 2B. Molecular modeling of the self-assembly of Azo-D-Lys-Phe-Ala using Materials Studio, showed consistent results on the morphology and microstructure of the fibrous network.

### Example 8

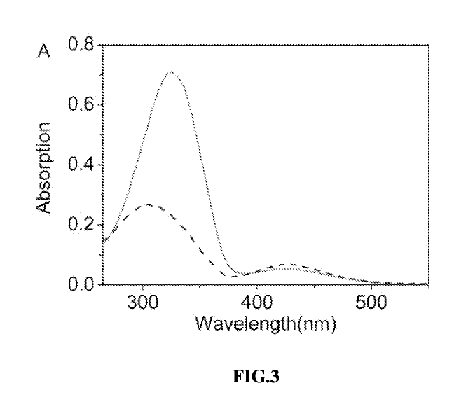

Photo-response of the hydrogels. The hydrogels formed by Azo-tripeptides exhibit a reversible photo-response. For example, the gel formed by Azo-Gln-Phe-Ala, has a photo-response in which a yellow gel in a container does not move in response to tipping of the container, and upon irradiation with UV light (365 nm) begins to turn to a liquid state within 3 minutes. Conversion of the hydrogel into a homogeneous non-viscous solution is complete in under 20 minutes, without external heating. The resultant non-viscous solution was then returned to the gel state upon ambient visible light irradiation for an average time span of two days. HPLC monitoring of the Azo-Gln-Phe-Ala gel before and after UV, photo-induced phase transition showed an increase in the ratio of cis- to trans- isomers. As shown in FIG. 3, for the sample corresponding to the original hydrogel before photoirradiation, the characteristic absorption of trans-azobenzene at about 330 nm predominated. After photo-irradiation and phase change, there was distinct decrease in this absorption and an increased absorption was observed at about 430 nm. The 430 nm absorption is characteristic of the cis-azobenzene. HPLC analysis on the components showed that the trans-Azo-Gln-Phe-Ala, was partially converted turned to cis-Azo-Gln-Phe-Ala after the photo-induced phase transition of the hydrogel. Hydrogels formed by several other tripeptides such as Azo-Gly-Phe-Ala and Azo-Lys-D-Phe-D-Ala also exhibit fast responses upon UV irradiation.

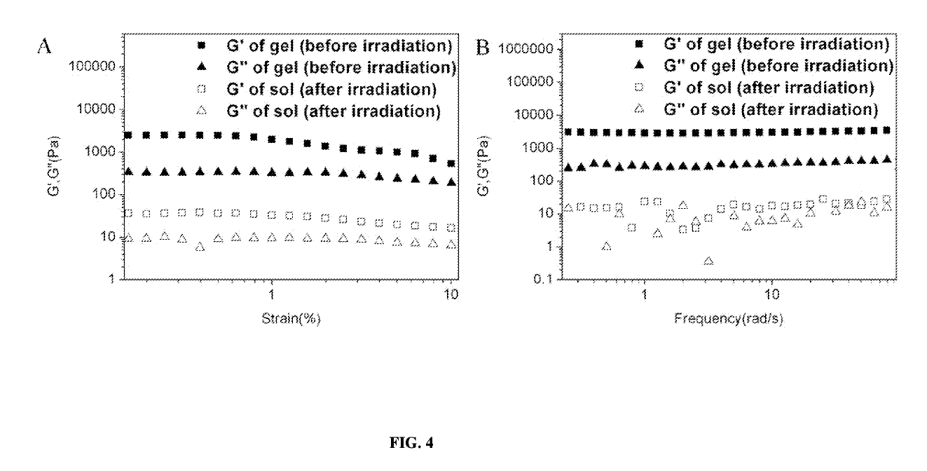

Changes in the viscoelastic properties of the hydrogel formed by Azo-Lys-D-Phe-D-Ala before and after UV irradiation has also been confirmed by oscillatory rheology as shown in FIG. 4. The dynamic strain sweep test (A) showed that storage modulus of the hydrogel before UV irradiation was 70 times higher than the one after UV irradiation. At the same time, the dynamic frequency sweep test (B) showed a greater than 100 times decrease in the storage modulus of the hydrogel after UV irradiation. It is noteworthy that photo-response of the hydrogels formed by azo-tripeptides was not uniform, and therefore those with the characteriatics appropriate for particular applications can be designed and selected.

Sensitivity of the hydrogels to UV-irradiation was shown in Table 1 as the total irradiation time needed for the gel began to collapse (tᵢₙᵢₜ). The Azo-Gly-Phe-Ala gel and Azo-Lys-D-Phe-D-Ala gel showed similar sensitivity (5 minutes and 7 minutes, respectively). The gel formed by Azo-Lys-Phe-Ala showed a partial phase transition after irradiation for 30 minutes. No photo-response was observed on the gel formed by Azo-Glu-Phe-Ala, even after 5 hours of UV-irradiation.

### Example 9

Hydrogels with multi-responses. One equivalent of vancomycin hydrochloride was added to the surface of an Azo-Lys-D-Phe-D-Ala gel. The yellow gel gradually changed to a homogeneous solution. A control experiment with Azo-Lys-Phe-Ala gel showed no phase change upon addition of vancomycin. This indicates that the response of Azo-Lys-D-Phe-D-Ala gel to vancomycin was due to a specific ligand-receptor interaction between the D-Phe-DAla motif and vancomycin. In addition to the response to ligand receptor interaction, the Azo-Lys-D-Phe-D-Ala gel also responds to heat and UV-irradiation. Upon heating to 42° C., the gel turned into a yellow solution, which upon cooling returns to the gel with the aid of ultrasonication. Gel to solution and vice versa could also be controlled through UV or visible light irradiation.

### Example 10

Azobenzene-linked bioactive short peptides. Structures of illustrative examples of bioactive short peptides linked with azobenzene are provided in Scheme 5.

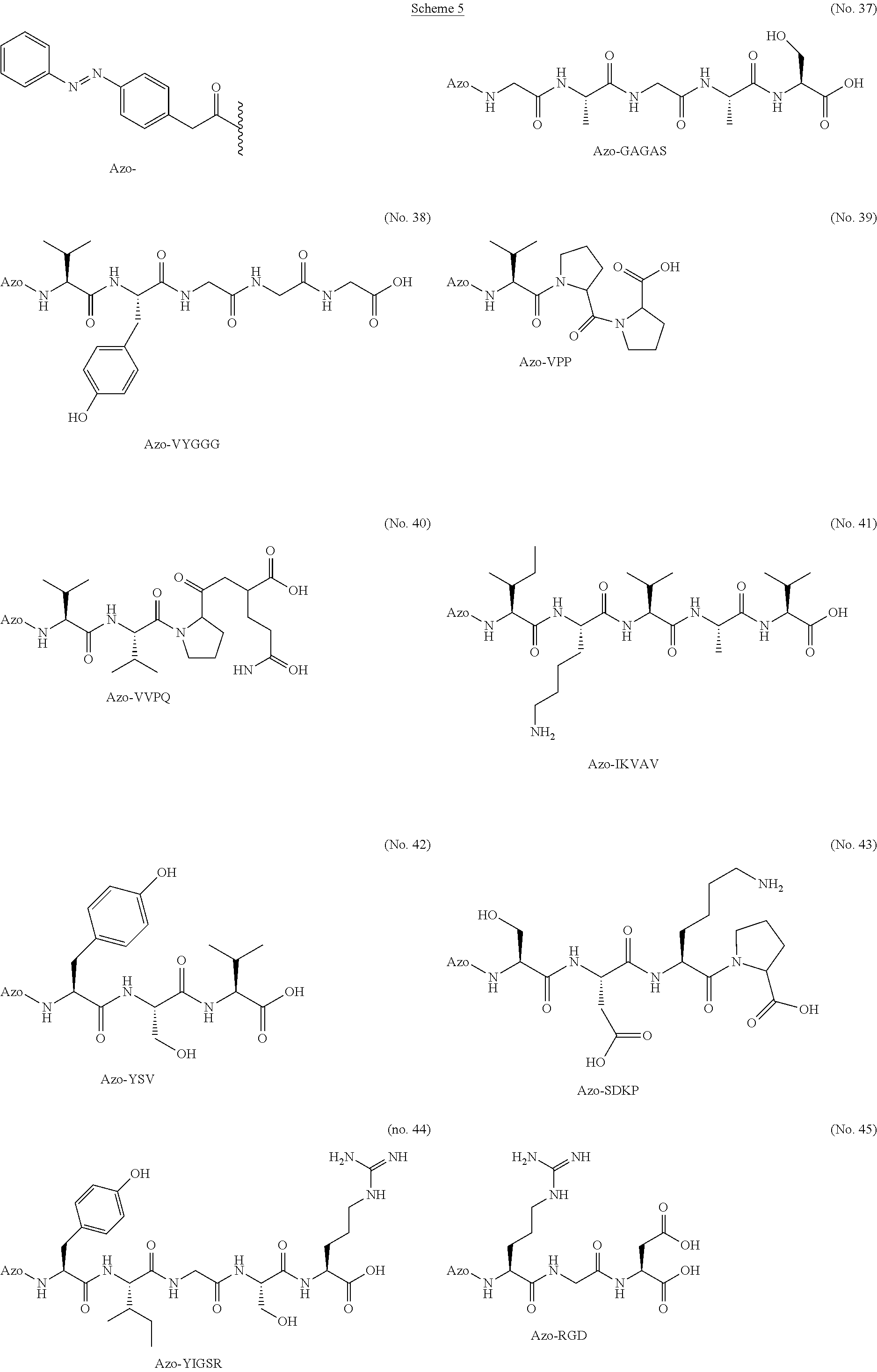

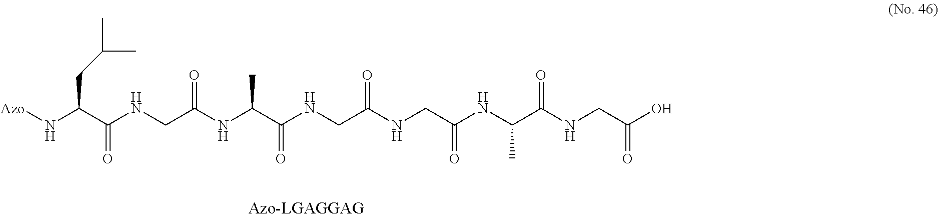

Conditions for the azobenzene substituted bio-active short peptides to form hydrogel are listed in Table 3.

TABLE 3
Bio-active Short Peptides, pH, and Lowest
Concentration To Form a Hydrogel.

| | | | C |
|---|---|---|---|
| No. | Azo-peptide | pH | (mg/mL) |
| 37 | Azo-GAGAS | 3.9 | 4.9 |
| 38 | Azo-VYGGG | 2.8 | 4.4 |
| 39 | Azo-VPP | — | — |
| 40 | Azo-VVPQ | — | — |
| 41 | Azo-IKVAV | 4.5 | 4.9 |
| 42 | Azo-YSV | 1.8 | 5.6 |
| 43 | Azo-SDKP | — | — |
| 44 | Azo-YIGSR | — | — |
| 45 | Azo-RGD | — | — |
| 46 | Azo-LGAGGAG | 1.6 | 6.7 |

### Example 11

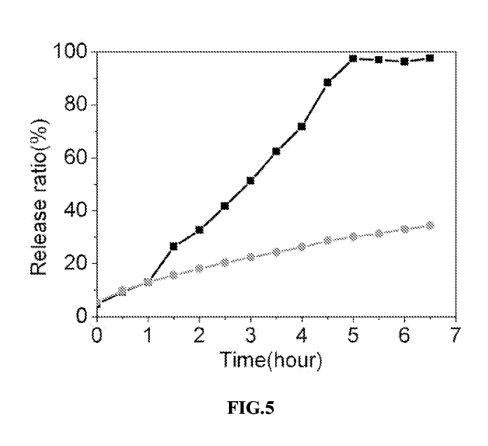

Application of the photo-responsive hydrogel in controlled release. FIG. 5 is an illustration of the quantitative comparison of the release ratio of vitamin B12 from Azo-Gln-Phe-Ala with (black square) and without (red dot) UV irradiation. The figure illustrates that the vitamin B12 is trapped in the hydrogel without disturbing the gel of the Azo-Gln-Phe-Ala. Without photoirradiation, the release of vitamin B12 is via concentration-gradient motivated diffusion from the gel to the upper layer water. Complete release, without irradiation, was greater than two days. Photo-irradiation of the mixture greatly accelerated the release process. The release ratio of vitamin B12 from the gel matrix to the upper layer water could be quantified through measuring the UV absorption of the solution at wavelength corresponding to the absorption of vitamin B12. UV absorption of the two parallel samples which had the same behavior within the first 1 hour without photo-irradiation was measured in due time courses. Once the sample was exposed to UV irradiation, a steep increase on the release ratio was observed as shown in FIG. 5. After 4 hours, a 97% photo-controlled release ratio was observed, much higher than the diffusion-controlled release of about 30%.

### Equivalents

While certain embodiments have been illustrated and described, it should be understood that changes and modifications can be made therein in accordance with ordinary skill in the art without departing from the technology in its broader aspects as defined in the following claims.

The use of the terms “a” and “an” and “the” and similar referents in the context of describing the elements (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Additionally the phrase “consisting essentially of” will be understood to include those elements specifically recited and those additional elements that do not materially affect the basic and novel characteristics of the claimed invention. The phrase “consisting of” excludes any element not specifically specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

Other embodiments are set forth in the following claims.

1. A composition comprising a stimuli-responsive compound represented by Formula I, or a salt thereof: 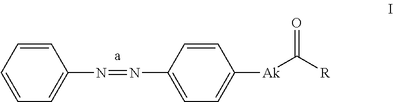
wherein:
a stereochemical conformation of the N═N double bond “a” is cis- or trans-;
Ak is an alkylene group; and
R represents a peptide comprising a series of two or more naturally occurring or synthetic amino acid residues.

2. The composition of claim 1, wherein R represents a peptide comprising from 2 to 20 naturally occurring or synthetic amino acid residues.

3. The composition of claim 1, wherein Ak is a C₁-C₁₀ alkylene group.

4. The composition of claim 1, wherein Ak is methylene, ethylene, or propylene.

5. The composition of claim 1, wherein R represents a peptide represented by: Xaa₁Xaa₂Xaa₃Xaa₄Xaa₅Xaa₆Xaa₇Xaa₈Xaa₉Xaa₁₀
wherein,
Xaa₁ is a naturally occurring or synthetic amino acid;
Xaa₂ is des-Xaa₂, or a naturally occurring or synthetic amino acid;
Xaa₃ is des-Xaa₃, or a naturally occurring or synthetic amino acid;
Xaa₄ is des-Xaa₄, or a naturally occurring or synthetic amino acid;
Xaa₅ is des-Xaa₅, or a naturally occurring or synthetic amino acid;
Xaa₆ is des-Xaa₆, or a naturally occurring or synthetic amino acid;
Xaa₇ is des-Xaa₇, or a naturally occurring or synthetic amino acid;
Xaa₈ is des-Xaa₈, or a naturally occurring or synthetic amino acid;
Xaa₉ is des-Xaa₉, or a naturally occurring or synthetic amino acid; and
Xaa₁₀ is des-Xaa₁₀, or a naturally occurring or synthetic amino acid.

6. The composition of claim 1, wherein at least Xaa₆, Xaa₇, Xaa₈, Xaa₉, and Xaa₁₀ are des-Xaa.

7. The composition of claim 1, wherein the stereochemical conformation of N═N double bond “a” is cis-.

8. The composition of claim 1, wherein the stereochemical conformation of N═N double bond “a” is trans-.

9. The composition of claim 1, wherein the stimuli-responsive compound is represented by Formula II, III, IV, or V; or a salt of Formula II, III, IV, or V: 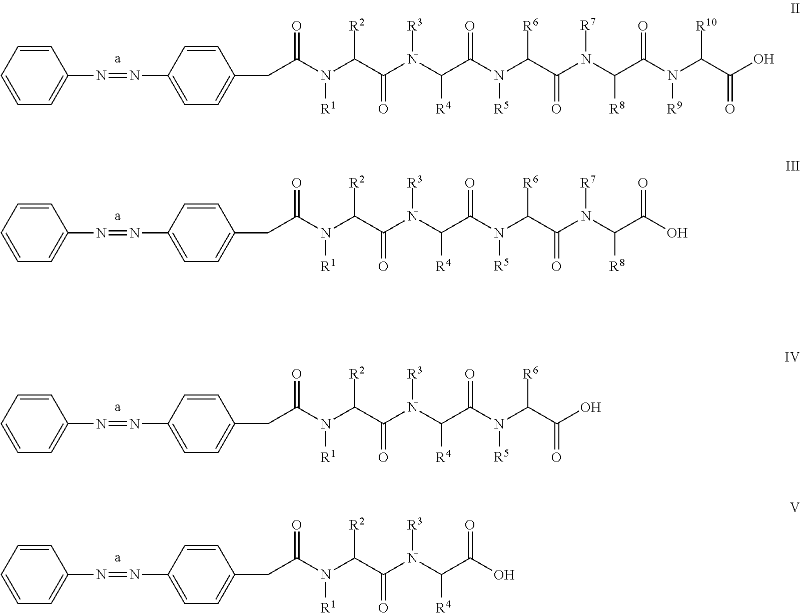
wherein:
a stereochemical conformation of N=N double bond “a” is cis- or trans-; and
each of r is independently absent or H, where n is 1, 3, 5, 7, or 9;
wherein:
if Rⁿ is H, then Rⁿ⁻¹ is H, OH, SH, COOH, COOR, alkyl, alkenyl, alkynyl, aryl, heterocyclyl, heteroaryl, aralkyl, amine, or amide; and
if one or more of Rⁿ is absent, the Rⁿ⁺¹ is alkylenyl and joins with the nitrogen of Nr to form a heterocycle.

10. The composition of claim 9, wherein R¹, R³, R⁵, R⁷ and R⁹ are each H.

11. The composition of claim 9, wherein R², R⁴, R⁶, R⁸ and R¹⁰ are independently H, C₁-C₈ alkyl, substituted C₁-C₈ alkyl, C₁-C₆ cycloalkyl, substituted C₁-C₆ cycloalkyl, phenyl, substituted phenyl, C₃-C₁₀ heterocyclyl, C₃-C₁₀ substituted heterocyclyl, C₃-C₁₀ heteroaryl, C₃-C₁₀ substituted heterocyclyl, or aralkyl.

12. The composition of claim 9, wherein R², R⁴, R⁶, R⁸ and R¹⁰ are independently H, methyl, ethyl, n-propyl, iso-propyl, 2-methylprop-1-yl, 1-methylprop-1-yl, n-butyl, CH₂OH, CH(CH₃)(OH), CH₂SH, CH₂CH₂SCH₃, CH₂(phenyl), CH₂(o-phenol), CH₂(m-phenol), CH₂(p-phenol), CH₂(indol-3-yl), CH₂C(O)OH, CH₂CH₂C(O)OH, CH₂C(O)NH₂, CH₂CH₂C(O)NH, CH₂(imidazole-5-yl), CH₂CH₂CH₂CH₂NH₂, CH₂CH₂CH₂NHC(NH)(NH₂), CH₂CH₂CH(OH)CH₂NH₂, CH₂CH₂CH₂CH₂NHCH₃, CH₂CH(COOH)COOH, CH₂OPO₃H₂, CH(CH₃)OPO₃H₂, CH₂CH₂OH, CH₂CH₂CH₂NH₂, CH₂CH₂CH₂NHC(O)NH₂, phenyl, or CH(CH₃)COOH.

13. The composition of claim 9, wherein one or more of Rⁿ are absent, and the Rⁿ⁺¹ joins with the nitrogen of Nr to form a C₃-C₆ heterocycle.

14. A method comprising:
providing a non-viscous solution comprising the composition of claim 1 and water;
exposing the non-viscous solution to visible light; and
maintaining the non-viscous solution in visible light for a first sufficient period of time to form a hydrogel.

15. The method of claim 14, wherein the first sufficient period of time is from about 1 hour to 5 days.

16. The method of claim 14 further comprising:
exposing the hydrogel to one or more of UV light, ultrasonication, or heat, and
maintaining exposure to one or more of UV light, ultrasonication, or heat, for a second sufficient period of time to collapse the hydrogel and form a collapsed non-viscous solution

17. The method of claim 1, wherein the second sufficient period of time is from about 10 seconds to about 1 day.

18. The method of claim 16, wherein upon the exposing to UV light, at least about 10% the compounds of Formula I, or the salt thereof in the composition are in the cis-configuration.

19. A controlled release composition comprising a pharmaceutically active agent and the composition of claim 1.

20. A process comprising:
providing a non-viscous solution comprising a pharmaceutically active agent, the composition of claim 1, and water; and
exposing the non-viscous solution to visible light for sufficient period of time for the composition to form a hydrogel;
wherein the hydrogel is a controlled release pharmaceutical composition.


SEQUENCE LISTING (7 sequences)

SEQ ID NO: 1 - protein, 5 aa, Artificial Sequence
  Description of Artificial Sequence Synthetic peptide
GAGAS

SEQ ID NO: 2 - protein, 5 aa, Artificial Sequence
  Description of Artificial Sequence Synthetic peptide
VYGGG

SEQ ID NO: 3 - protein, 4 aa, Artificial Sequence
  Description of Artificial Sequence Synthetic peptide
VVPQ

SEQ ID NO: 4 - protein, 5 aa, Artificial Sequence
  Description of Artificial Sequence Synthetic peptide
IKVAV

SEQ ID NO: 5 - protein, 4 aa, Artificial Sequence
  Description of Artificial Sequence Synthetic peptide
SDKP

SEQ ID NO: 6 - protein, 5 aa, Artificial Sequence
  Description of Artificial Sequence Synthetic peptide
YIGSR

SEQ ID NO: 7 - protein, 7 aa, Artificial Sequence
  Description of Artificial Sequence Synthetic peptide
LGAGGAG